(19) United States
(12) Patent Application Publication
(10) Pub. No.: US 2022/0157431 A1
(43) Pub. Date: May 19, 2022

(54) MIRROR-NEURONS STIMULATION

(71) Applicant: Roy Joshua Sarfati, Hallandale Beach, FL (US)

(72) Inventor: Roy Joshua Sarfati, Hallandale Beach, FL (US)

(21) Appl. No.: 17/108,791

(22) Filed: Dec. 1, 2020

(51) Int. Cl. G16H 20/60

(52) U.S. Cl. CPC G16H 20/60

(57) ABSTRACT

An innovative multi-dimensional, non-invasive, integrative digital habilitation and rehabilitation treatment programs in identifying and treating cognitive, memory, speech, language, and communication disorders associated with various neurological etiologies. The programs focus and utilize systematic and progressive activation of the Mirror Neurons System in coordination with the level of complexity. The structure of exercises consists of coordinating the production of observed activities delivered by synchronous, audio/visual interactive digital exercises conveyed by special computerized systems. The delivery of program is provided both in clinical setting and by telemedicine module. Upon completion of the treatment phase, the person with the neurological condition is placed on self-supervised exercises critical in the maintenance and retention of clinical gains.

### BACKGROUND OF INVENTION

### 1. Field of Invention

The present invention relates to the treatment or therapy of speech and language disorder. In particular, the present invention is a fully-integrated program in the activation of mirror neurons for speech and language disorders of neurogenic origin such as but not limited to patients with Alzhiemer's, Parkinson's, Dementia, Stroke, and Autism.

### 2. Prior Art

No prior art was found relating to the activation of mirror neurons and its inclusion in the treatment or therapy for speech and language disorders.

### SUMMARY OF INVENTION

The present invention is multi-dimensional treatment program for persons who are afflicted with neurogenic medical conditions and consequently, suffer from speech and language disorders. This fully integrative therapy program is and but not limited to persons with Alzhiemer's, Parkinson's, Dementia, Stroke, and Autism. The treatment program focuses on the activation of Mirror Neurons System located in the premotor cortex, the supplementary motor area, the primary somatosensory cortex, and the inferior parietal cortex.

The exercises are formulated to first observe the targeted activity and imitate the desired action. While the double evoked imitation takes place, the mirror neuron system is activated and increasing in number which is essential in restoring the link between comprehension, cognition, and verbal expression. The activation of mirror neurons system is critical in developing alternate neural pathways for successful interaction between the aforementioned components that impact speech and language.

The present speech-language treatment program for patient with neurological disorders is employed by medically trained personnel such as a speech-language pathologist.

The interactive, multi-faceted program contains progress measurement system with periodic review of accomplishment overseen by medically trained personnel such as the speech-language pathologist. The hands on, progressive program is delivered to target cognition, memory, expressive communication, comprehension, and speech deficits resulting from neurological injuries.

The multi-faceted program consists of standalone animations, animation interactions with third parties, and animated synchronous audio/visual and verbal interaction for comprehensive and systematic stimulation of deficits. The progressive training or exercise program has series of phases or stages of increasing complexity with anticipated results.

The hands-on progressive, multi-faceted training or exercise program is delivered in an intensive-concentrated format. The exercise day is preferably divided in sessions in accordance to the needs of the patients. The preferred schedule may be changed due to various factors but not exclusive to the medical status of condition of the patient.

Upon completion of the progressive, multi-faceted program, the patient enters a self-administered phase of the program, designed for maintaining the gains achieved during the active, hands-on phase of the program. Periodic review of the patient's performance is conducted by the medical team.

### OBJECTS OF THE INVENTION

It is an object of the invention to provide formulate and carry out an individualized, personalized program for overcoming speech-language impairments due to neurological injuries.

Another object of the invention is to address the physiological aspect of cognitive disorder including memory dysfunction and cognitive coordination associated with neurological impairment(s) using non-invasive techniques designed for cortical reorganization that are tailored to the patient's needs and implementation is based on a viable schedule peculiar to the patient.

A further object of the invention is to address the physiological aspect of expressive communication disorder associated with neurological impairment using double-imitation evoked exercises non-invasive in nature. The treatment based on the needs of the particular patient.

It is another object of the invention to address the physiological aspect of language comprehension disorders associated with the neurological impairment peculiar to the patient using associative cognitive-linguistic tasks non-invasive in nature. The implementation of the treatment protocol is established based on the patient's needs.

A still further object of the invention is to provide a visual-audio interactive system during active exercises in a digitalized manner. The exercises comprise of animated synchronous audio-visual-verbal interaction. The complexity of exercises increases once a patient achieved a performance criterion level indicative of mastery.

It is another object of the invention to provide an improved process for providing a corrective audio-visual feedback system in a therapeutic neuro-cognitive linguistic rehabilitation program. Correct response to active therapeutic exercise is provided in a digitalized auditory format.

Another object of the invention is to provide a personal rehabilitation correction program that incorporates a fully integrated, multi-therapeutic program with professional team review of patient self-evaluation of the effectiveness of self-treatment during the post-treatment preventive maintenance phase of the neuro-cognitive linguistic rehabilitation and/or habilitation program.

A further object of the invention is to provide a personalized neuro-cognitive linguistic program characterized by hands-on active treatment phase and a post active treatment, self-disciplined phase which includes self-administered review of self-evaluation by professional personal and updating of self-administered activity for preventive maintenance against multi-factorial regression to patient's neuro-cognitive linguistic post-treatment achievement status.

### BRIEF DESCRIPTION OF THE DRAWINGS

FIG. 1 is a flow chart representing the phases of the multi-dimensional, non-invasive, integrative habilitation and rehabilitation program.

FIG. 2 is a flow chart representing in more detail, the Multi Faceted Diagnostic Procedure analytics process represented in FIG. 1;

FIG. 3 is a flow chart representing, in more detail, the Exercise phase, represented in FIG. 1;

FIG. 3a is a flow chart representing, in more detail, the Exercise Application and Interaction represented in FIG. 3;

FIG. 3b is a flow chart representing, in more detail, Exercise Configuration Updates and Data Storage represented in FIG. 3;

FIG. 4 is a flow chart representing the Post-Treatment Retention Program and Interaction.

FIG. 5 is a flow chart representing the interaction between a patient and center during the treatment and post-treatment phase.

### DETAILED DESCRIPTION OF THE INVENTION

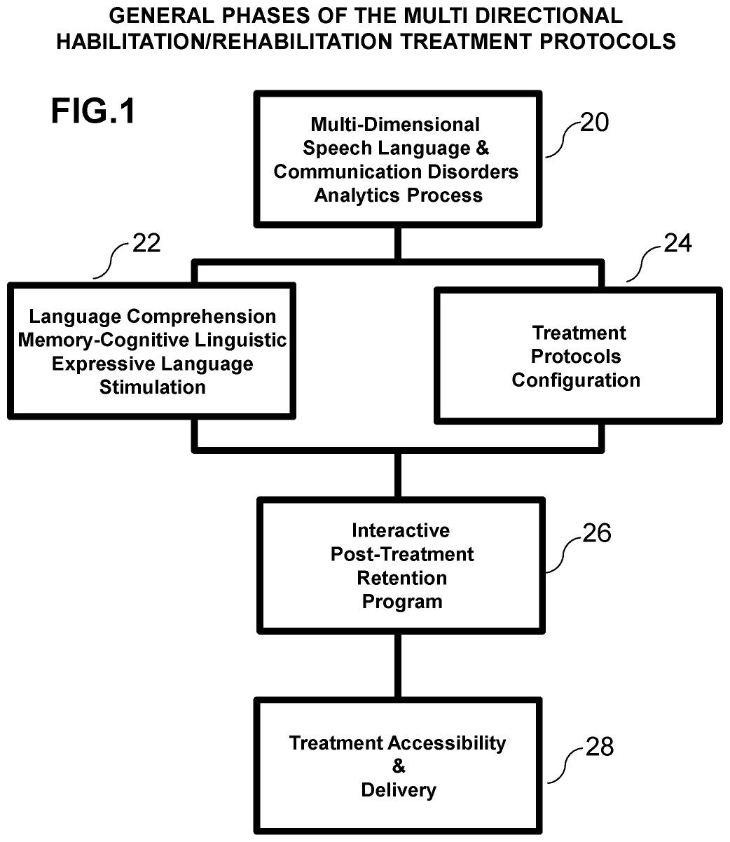

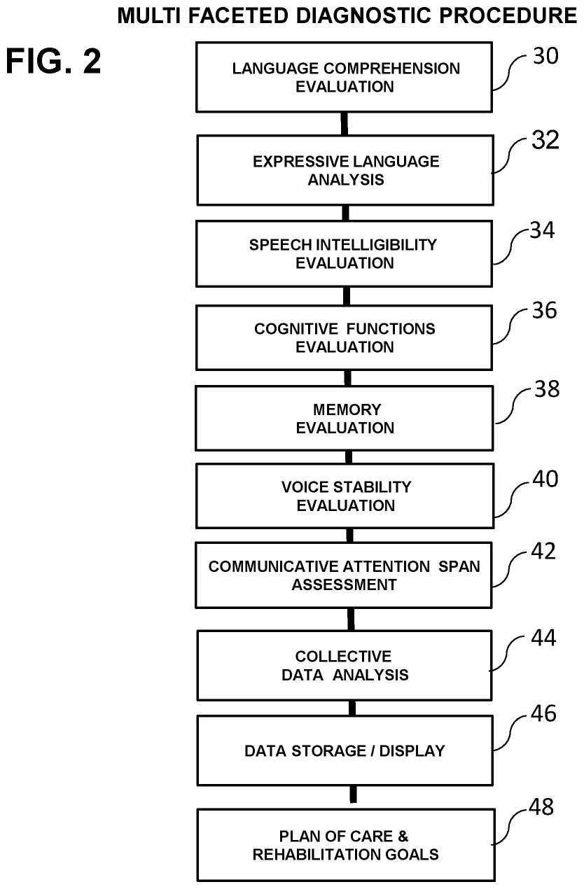

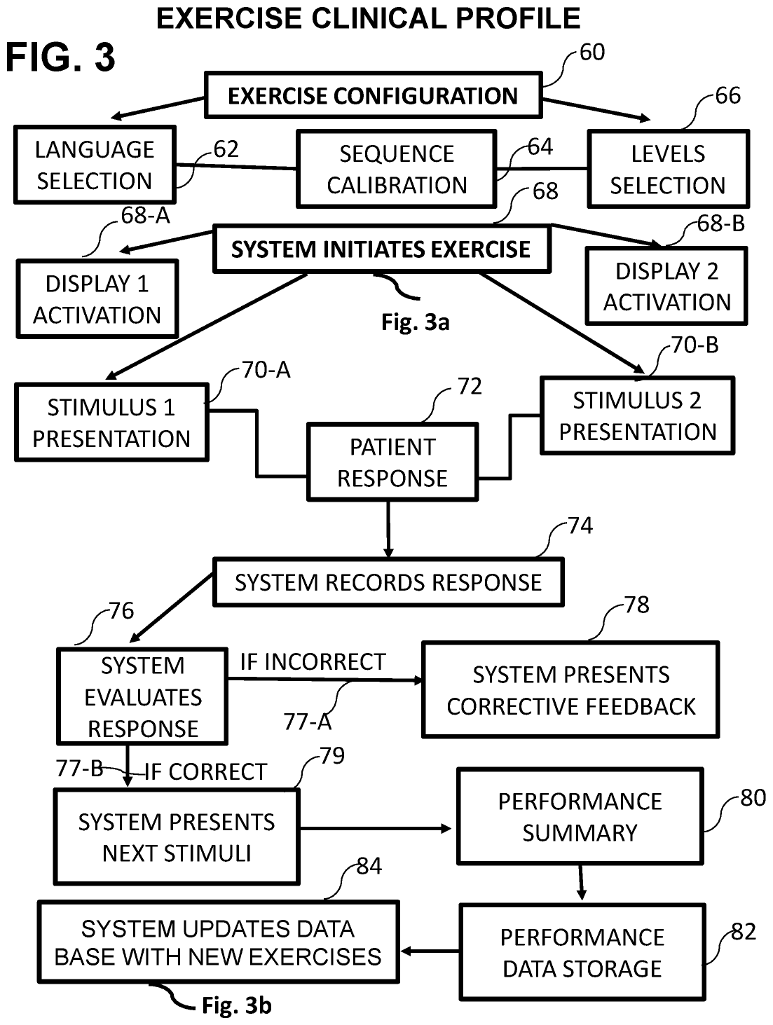

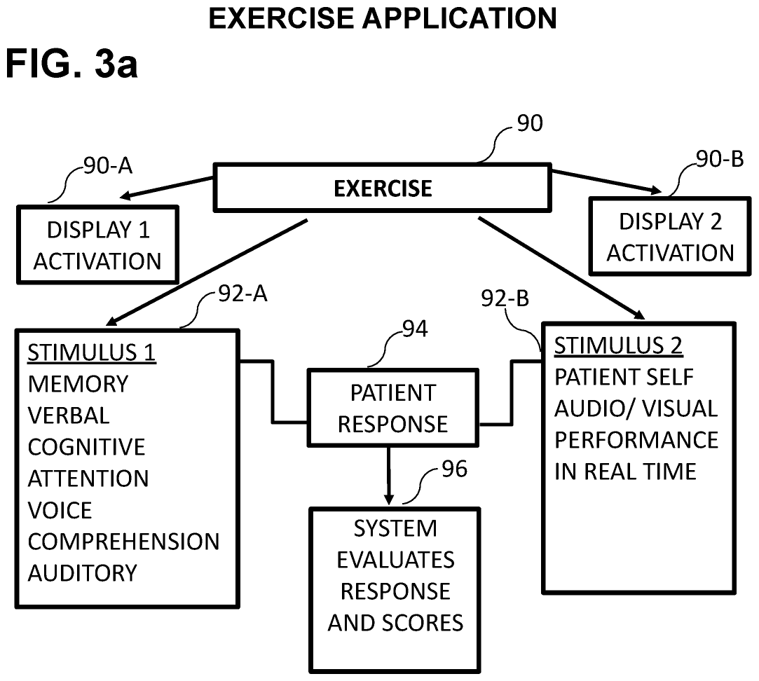

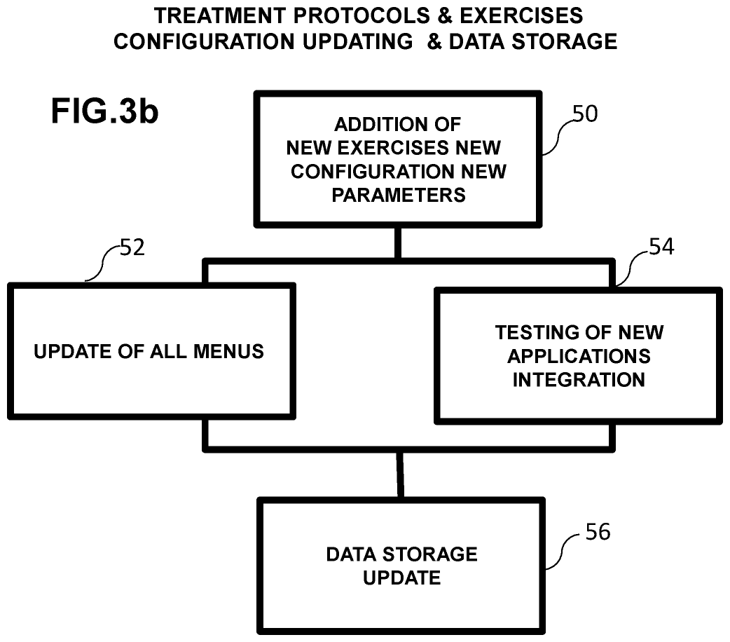

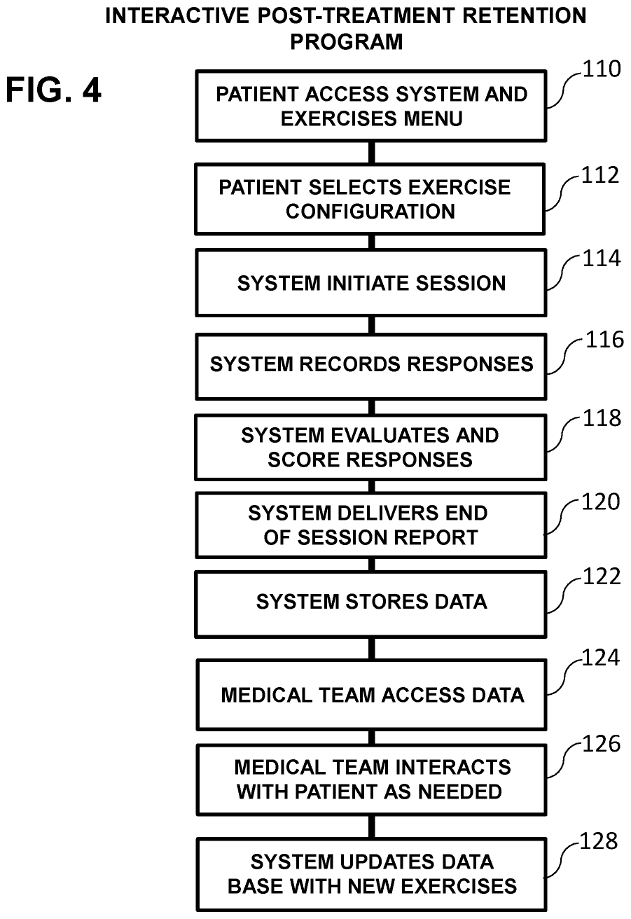

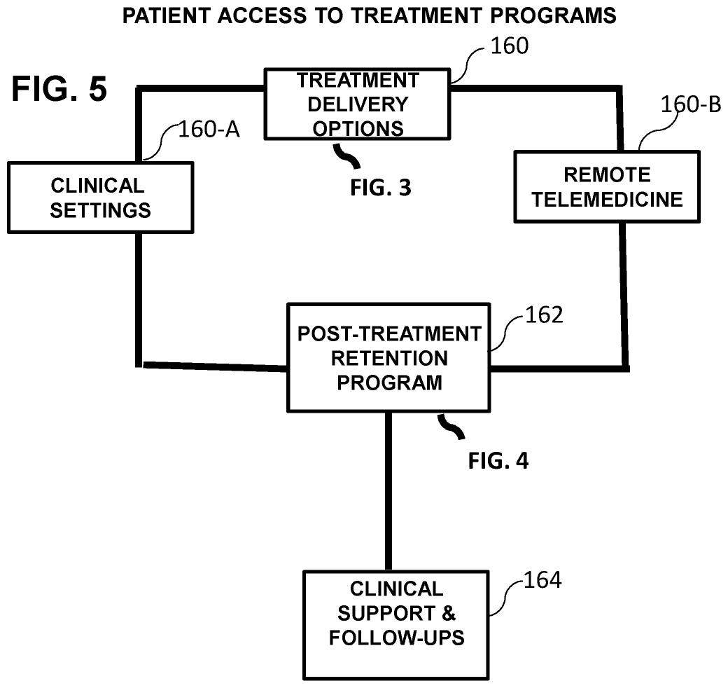

The flow chart presented in FIG. 1 represents the general habilitation/rehabilitation treatment protocols. Each step represented in the the flow chart FIG. 1 is more fully defined in the FIGS. 2, 3, 3a, 4, and 5. Block 20, Multi-Dimensional Speech Language & Communication Disorders Analytics Process is more fully represented and/or defined by the flow chart in FIG. 2. Block 22, Language Comprehension Memory-Cognitive Linguistic Expressive Language Stimulation is more fully represented and/or defined by the flow chart FIGS. 3 and 3a. Block 24, TREATMENT PROTOCOLS CONFIGURATION is more fully represented and/or defined by the flow chart in FIG. 3b. Block 26, INTERACTIVE POST-TREATMENT RETENTION PROGRAM is more fully represented and/or defined by the flow chart in FIG. 4. Block 28, is more fully represented and/or defined by the flow chart in FIG. 5.

As represented by the flow chart in FIG. 2, the MULTI-FACETED DIAGNOSTIC PROCEDURE, block 20, is defined by a protocol where a patient initially goes through a comprehensive series of evaluation and analysis of cognitive, speech and language components affecting communication of the patient. The objective of LANGUAGE COMPREHENSION EVALUATION, block 30 is to analyze verbal language processing at concrete and abstract information level for determining the patient's understanding of information including but not limited to discourse. The objective of EXPRESSIVE LANGUAGE ANALYSIS, block 32 is to evaluate and analyze the presence of constraints if any of verbal output and formulated utterances with syntactic and semantic cohesion. The objective of SPEECH INTELLGIBILITY EVALUATION, block 34 is to analyze and evaluate the production of speech sounds embedded in utterances versus target speech sounds. The objective of COGNITIVE FUNCTION EVALUATION, block 36 is to analyze and evaluate cognitive associative skills, concrete and abstract judgement skills as well as concrete and abstract problem-solving skills. The objective of MEMORY EVALUATION, block 38 is to assess working memory in the immediate and delayed response format. The objective of VOICE STABILITY EVALUATION, block 40 is to define and analyze the vocal pitch, vocal loudness, and vocal quality of the patient, for determining vocal cord function and if any, vocal perturbation. The objective of COMMUNICATIVE ATTENTION SPAN ASSESSMENT, block 42 is to analyze and define the patient's attention in terms of sustained attention, selective attention, alternating attention, and divided attention.

The results of the assessments are evaluated, defining the following clinical results;

Language comprehension;

Expressive Language;

Speech Intelligibility;

Concrete reasoning;

Abstract reasoning;

Associative relations;

Judgment conflict resolution;

Memory;

Vocal pitch;

Vocal intensity;

Vocal frequency;

Vocal cycle Periodicity/Aperiodicity pattern;

Sustained attention;

Selective attention;

Alternating attention;

Divided attention;

The results of the several examinations and analyses are recorded and stored. The objective of COLLECTIVE DATA ANALYSIS, block 44 is to process the stored diagnostic date, to provide analytics, and define the patient's profile with the assistance of a computer programmed to convey the diagnostic profile of a patient. The objective of DATA STORAGE/DISPLAY, block 46 is to establish storage of the analytics using database specific to elicited diagnostic information. The objective of PLAN OF CARE & REHABILITATION/HABILITATION GOALS, block 48 is to gather all the diagnostic date for determining the course treatment identifying the critical areas to be addressed using a computer programmed designed to formulate report of said patient status.

FIG. 3 represents and/or defines more completely, block 22, the EXERCISE CLINICAL PROFILE of the multi-faceted speech, language, cognitive habilitation and rehabilitation program FIG. 1. The objective of EXERCISE CONFIGURATION, block 60 is to setup the clinical configuration in terms of language selection, assigning the sequence of therapeutic exercises, and the level of complexity determined by a patient's diagnostic profile. The objective of SYSTEM INITIATES EXERCISE, block 68 is defined by the activation of the computerized program presenting the stimuli to patient. Block 68 is more fully defined in the flow chart in FIG. 3a. The objective of SYSTEM RECORDS RESPONSE, block 74 is to capture, process, and analyze response. Correct response advances the patient to the next stimulus. Incorrect response triggers the Corrective Feedback mechanism integrated in the computerized program. At end of an exercise, the system provides and stores a patient's performance.

The focus is directed to FIG. 3a, which is a flow chart defining more extensively, the EXERCISE APPLICATION, block 68 in FIG. 3.

The active exercise program is a series of multi-faceted exercises targeting double evoked mirror neuron stimulation. Blocks 90a and 90b represent synchronous display of stimuli on dual monitors. Display 1 is defined as the monitor exhibiting the stimuli relating to the critical areas of treatment: memory, cognition, attention, expressive communication, language comprehension, voice, and speech in which these elements are presented in block 92. Display 2 is characterized as the reflection monitor as it functions as a Mirror that reflects the patient's verbal and visual responses triggering the activation of the Mirror Neurons. Block 94, PATIENT RESPONSE, represents the system recording the particular patient's selection and verbal utterances. Block 96, SYSTEM EVALUATES RESPONSE AND SCORES, is defined as the portion of the program in which the registered responses are compared to a set of acceptance criteria. Based on the overall recorded responses, a score is calculated,

The present Mirror Neuron Stimulation multi-dimensional, non-invasive, integrative habilitation and rehabilitation treatment program positively includes a post-treatment retention program to preclude regression of progress and ensure long-term stability. This is represented by the flow chart FIG. 4, INTERACTIVE POST-TREATMENT RETENTION PROGRAM. The Interactive Post-Treatment Retention program provides those patients access to the System and its Exercise Menu, block 110. FIG. 5, PATIENT ACCESS TO TREATMENT PROGRAMS, addresses the various modes of accessing the exercise menu in the clinic, block 160 or by remote telemedicine, block 162.

Once the exercise menu has been selected, the said patient chooses the appropriate exercise configuration, block 112. Then, the system initiates practice session, block 114, and records the responses, block 116. The responses are then analyzed and a score is produced during the interaction with the system, block 118. After the said patient completes the treatment session, the system generates the end of session report, block 120. The said patient's performance data is collected and stored, block 122. The medical team attains the said individual clinical records reviewing the performance of said individual, block 124, and corresponds with the said patient on a need basis, block 126. Periodically, updates are delivered to database with new exercises, block 128.

FIG. 5 represents and/or defines modalities of treatment accessibility between the patient and the Clinic. Block 160 defines the patient's treatment accessibility options which consist of face to face interaction in the Clinic, block 160-A or via remote telemedicine, block 160-B, provided over a secured broadband platform in real time, face-to-face interaction in synchronous audio/visual format. The treatment is described in FIG. 3.

Block 162 defines post-treatment retention program described in FIG. 4. various follow-up communications between the patient and the Clinic. Block 164 represents clinical support and follow-ups with patients will conducted with emails, phone, and telemedicine.

In the foregoing description of the the invention, referenced to the drawings, certain terms have been used for conciseness, clarity, and comprehension. However, no unnecessary limitations are to be implied from or because of the terms used, beyond the requirements of the prior art, because such terms are used for descriptive purposes and are intended to be broadly construed. Furthermore, the description and illustration of the invention are by way of example, and the scope of the invention is not limited to the exact details shown, represented, or described.

Having now described a preferred embodiment of the invention, in terms of features, discoveries, and principles, along with certain alternative construction and suggested changes, other changes that may become apparent to those skilled in the art may be made, without departing from the scope of the invention defined in the appended claims.

What is claimed is:

1. A method for Mirror-Neurons Stimulation in the treatment of speech, language, memory, cognitive, and communication disorders for individuals who are afflicted with developmental and/or neurological medical conditions, consisting of the following steps of:
a) performing a multi-faceted diagnostic procedure on said individuals including:
i. a language comprehension evaluation
ii. an expressive language analysis
iii. speech intelligibility evaluation
iv. cognitive functions evaluation
v. memory evaluation
vi. voice stability evaluation
vii. communicative attention span assessment
for attaining data on the individual who is afflicted with developmental and/or neurological medical conditions for processing and collective data analysis and storage. Then, the formulation of Plan of Care and Habilitation/Rehabilitation Goals for said individual.
b) performing a series of supervised speech, language, cognitive, and memory exercises in isolation or in combination in accordance to the Plan of Care, and series of stimuli exercises targeting mirror neuron stimulation via double evoked imitation, each supervised exercise in each series being progressively increased in complexity, while simultaneously monitory, and adjusting the elicited responses of the said patient, for gaining targeted skills; and
c) performing double-evoked elicitation exercises for mirror neurons stimulation, defining speech, language, cognitive, and memory post-treatment retention program designed for preventative and restoring regression of said patient.

2. A method as in claim 1 and further including the step of:
d) analyzing the effectiveness of said patient-administered speech, language, cognitive, memory, and communication exercises as stated in the Plan of Care.

3. A method as in claim 2 and further including the step of:
e) re-evaluating said patient-administered speech, language, cognitive, memory, and communication exercises as defined the performance of exercises for said patient.

4. A method as in claim 3 and further including the step of:
f) Updating said patient-administered speech, language, cognitive, memory, and communication exercises.

5. A method for surmounting speech, language, cognitive, memory, and communicative challenges in a patient comprising the steps of:
a) performing a multi-faceted diagnostic procedure on said individuals including
i. a language comprehension evaluation,
ii. an expressive language analysis,
iii. speech intelligibility evaluation,
iv. cognitive functions evaluation,
v. memory evaluation,
vi. voice stability evaluation,
vii. communicative attention span assessment,
for obtaining data on said patient for formulating a habilitation or rehabilitation program for targeted speech, language, memory, cognitive, and/or other communicative problems for attaining effective speech and/or communicative skills for said patient;
b) activating the Mirror Neurons of said patient by stimulating double evoked imitation of said patient;
c) providing a series of double evoked imitation and stimuli exercises for said patient, said exercises defined by said habilitation/rehabilitation program, and said series of attended exercises being progressively increased in speech, language, memory, cognitive, and other communicative complexity;
d) monitoring and adjusting the therapeutic program of said patient with performance of step c); and
e) performing a self-supervised speech, language, cognitive, memory, and other communicative disorders retention exercises for maintaining speech, language, memory, cognitive, and other communicative gains of said patient.

6. A method as in claim 5 and further including the step of;
f) self-monitoring said self-supervised speech, language, memory, cognitive, and other communicative retention exercises for defining an effectiveness of said self-supervised speech, language, memory, cognitive, and other communicative retention exercises on said patient.

7. A method as in claim and further including the step of;
g) self-analyzing speech, language, memory, cognitive, and other communicative skills of said patient for evaluating the level of speech, language, memory, cognitive, and other communicative gains maintained after performance of self-supervised speech, language, memory, cognitive, and other communicative exercises for providing a preventative retention program for speech, language, memory, cognitive, and other communicative skills.

8. A method as in claim 6 and further including the steps of;
g) self-analyzing speech, language, memory, cognitive, and other communicative skills of said patient for self-evaluating the level of speech, language, memory, cognitive, and other communicative skills retained for determining the presence and severity of speech, language, memory, cognitive, and other communicative regression; and
h) consulting with clinical specialists who developed said habilitative/rehabilitative therapeutic program of analyzing said level of speech, language, memory, cognitive, and communicative skills evaluated by said patient for receiving updated said self-supervised retention program for the prevention of speech, language, memory, cognitive, and other communicative regression.

9. A method as in claim 6 and further including the step of;
g) reporting self-analysis of speech, language, cognitive, memory, and other communicative gains and/or regression to the clinical specialists who formulated said habilitative/rehabilitative program.

10. A method as in claim 9 and further including the step of;
h) revising said self-supervised speech, language, cognitive, memory, and other communicative exercises for preventing regression of speech, language, cognitive, memory, and other communication skills.

11. A method as in claim 9 and further including the step of;
h) updating said self-supervised speech, language, cognitive, memory, and other communication exercises for the prevention of speech, language, cognitive, memory, and other communicative regression of said patient and communicating said updates to said patient.

12. A method as in claim 5 and further including the step of; f) self-monitoring said self-supervised speech, language, cognitive, memory, and other communication retention exercises and self-analyzing retained speech, language, cognitive, memory, and other communicative skills for determining the effectiveness of said self-supervised speech, language, cognitive, memory, and communicative retention exercises of said patient.

13. An innovative habilitation/rehabilitation treatment program for attaining speech, language, memory, cognitive, and other communicative skills via Mirror-Neurons Stimulation using double evoked imitation for persons who are afflicted with developmental and/or neurogenic medical conditions. The said habilitation/rehabilitation treatment program provides for multi-faceted diagnostic protocols for said patients. The program consists of exercises progressively increasing complexity for achieving speech, language, cognitive, memory, and other communicative skills detailed in the Plan of Care, the therapeutic habilitation/rehabilitation program, including the steps of:
a) providing a self-administered retention exercise program for maintaining speech, language, cognitive, memory, and other communication skills attained during the said supervised, hands-on program; and
b) providing for self-evaluation of speech, language, cognitive, memory, and other communicative skills retained during said self-administered retention exercise program for determining effectiveness of said self-administered speech, language, cognitive, memory, and other communicative skills retention program against regression.

14. The innovative habilitation/rehabilitation treatment program method as in claim 13 for attaining speech, language, cognitive, memory, and other communicative skills for developmental and/or neurogenic medical conditions and further including the steps of:
c) analyzing said self-evaluation for determining speech, language, memory, cognitive, and other communicative gains; and
d) updating said self-administered speech, language, cognitive, memory, and other communicative exercises against regression.

15) An innovative habilitation/rehabilitation treatment method as in claim 14, and further including the steps of:
e) communicating said updated self-administered speech, language, cognitive, memory, and other communicative exercises to said person for self-supervised exercise.

16) A therapeutic habilitation/rehabilitation program using Mirror-Neuron Stimulation for overcoming speech, language, memory, cognitive, and other communicative disorders resulting from developmental and/or neurogenic medical conditions, comprising the steps of:
a) performing a multi-faceted diagnostic procedure on said individuals including
i. a language comprehension evaluation,
ii. an expressive language analysis,
iii. speech intelligibility evaluation,
iv. cognitive functions evaluation,
v. memory evaluation,
vi. voice stability evaluation,
vii. communicative attention span assessment,
for obtaining data on said patient for formulating a habilitation or rehabilitation program for targeted speech, language, memory, cognitive, and/or other communicative problems for attaining effective speech and/or communicative skills for said patient;
b) activating the Mirror Neurons of said patient by stimulating double evoked imitation of said patient;
c) providing a series of double evoked imitation and stimuli exercises for said patient, said exercises defined by said habilitation/rehabilitation program, and said series of attended exercises being progressively increased in speech, language, memory, cognitive, and other communicative complexity;
d) monitoring and adjusting the therapeutic program of said patient with performance of step c); and
e) performing a self-supervised speech, language, cognitive, memory, and other communicative disorders retention exercises for maintaining speech, language, memory, cognitive, and other performing a multi-faceted diagnostic procedure on said individuals including
i. a language comprehension evaluation,
ii. an expressive language analysis,
iii. speech intelligibility evaluation,
iv. cognitive functions evaluation,
v. memory evaluation,
vi. voice stability evaluation,
vii. communicative attention span assessment,
for obtaining data on said patient for formulating a habilitation or rehabilitation program for targeted speech, language, memory, cognitive, and/or other communicative problems for attaining effective speech and/or communicative skills for said patient;
f) activating the Mirror Neurons of said patient by stimulating double evoked imitation of said patient;
g) providing a series of double evoked imitation and stimuli exercises for said patient, said exercises defined by said habilitation/rehabilitation program, and said series of attended exercises being progressively increased in speech, language, memory, cognitive, and other communicative complexity;
h) monitoring and adjusting the therapeutic program of said patient with performance of step c); and
i) performing a self-supervised speech, language, cognitive, memory, and other communicative disorders retention exercises for maintaining speech, language, memory, cognitive, and communicative gains of said patient. communicative gains of said patient.
